(19) United States
(12) Patent Application Publication
(10) Pub. No.: US 2008/0140012 A1
(43) Pub. Date: Jun. 12, 2008

(54) HEMODIALYSIS GRAFT

(76) Inventor: Oswald S. Binford, Corsicana, TX (US)

Correspondence Address: Michael A. O'Neil;Michael A. O'Neil, P.C., Suite 820, 5949 Sherry Lane, Dallas, TX, 75225, US

(21) Appl. No.: 11/457,044

(22) Filed: Jul. 12, 2006

(51) Int. Cl. A61M 5/32

(52) U.S. Cl. 604/175; 604/8; 606/153; 623/1.1

(57) ABSTRACT

The curved section of a hemodialysis graft comprises an inner interior surface defined by a relatively large radius and an outer interior surface defined by a relatively small radius for maintaining laminar flow of blood passing through the curved section of the graft and thereby substantially reducing cellular proliferation and blood clotting.

### TECHNICAL FIELD

This invention relates generally to the construction of hemodialysis grafts, and more particularly to an improved hemodialysis graft construction which substantially eliminates cellular proliferation and clotting of blood flowing therethrough.

### BACKGROUND AND SUMMARY OF THE INVENTION

Hemodialysis comprises the use of a device called a dialyzer to clean wastes from blood when the patient's kidneys fail to do so. Blood is removed from the patient's body and directed through the dialyzer which removes wastes and excess fluid. The cleaned blood is then returned to the patient's body. Without dialysis treatment, the patients will die.

FIG. 1 illustrates a graft G connected between an artery and a vein of a patient P. Other types and kinds of graft placements are well known to those skilled in the art. Regardless of where it is placed, the function of the graft G is to facilitate withdrawal of blood from the patient P for cleaning in a dialyzer and for returning the cleaned blood to the patient P.

Heretofore grafts utilized in hemodialysis have been circular in cross section. Conventional hemodialysis grafts tend to clog with a proliferation of cells and coagulated blood. When this occurs the graft must be surgically declotted or a new graft must be installed at a different location. Graft declotting and replacement are surgical procedures meaning that the patient must undergo repeated surgeries simply to assure a flow of blood through the graft adequate to facilitate hemodialysis.

Virtually every hemodialysis graft is curved to a greater or lesser degree. When blood flows through the curved portion of a hemodialysis graft the portion thereof flowing through the outside portion of the curve flows at a different rate as compared with the flow of blood through the inside portion of the curve thereby resulting in turbulence. It is accepted by the medical community that turbulence within the graft, as documented by doppler ultrasound, predisposes the graft to failure. It is theorized that the turbulence within the graft traumatizes the inner wall of the blood vessel at the vein-graft junction, commonly referred to as the venous anastamosis. This inner wall of the vein is composed of endothelial cells. In response to this trauma, the endothelial cells proliferate into the lumen of the graft. Proliferation of the endothelium narrows the lumen in the vicinity of the venous anastamosis thereby increasing the turbulence within the graft and decreasing the blood flow rate within the graft. The increased turbulence results in additional endothelial trauma and subsequent endothelial proliferation. This cumulative process continues until the diminished blood flow within the graft is no longer suitable for hemodialysis. Without surgical intervention, a blood clot forms throughout the graft due to stagnant blood flow and the patient must have a new graft installed.

The present invention comprises a hemodialysis graft which substantially reduces turbulence in blood flowing therethrough. Because turbulence is substantially eliminated stimulation for endothelial proliferation within the graft is markedly reduced and the tendency of blood flowing through the hemodialysis graft of the present invention to clot is markedly reduced. This is in turn substantially extends the useful life of the graft which in turn results in a significant reduction in the number of surgeries that the patient must endure during hemodialysis treatment.

### BRIEF DESCRIPTION OF THE DRAWINGS

A more complete understanding of the present invention may be had by reference to the following Detailed Description when taken in connection with the accompanying Drawings, wherein:

FIG. 1 is a diagrammatic illustration of a human arm having a hemodialysis graft installed thereon;

FIG. 2 is a perspective view illustrating the hemodialysis graft of the present invention; and

FIG. 3 is a sectional view taken along the line 3-3 in FIG. 2 in the direction of the arrows.

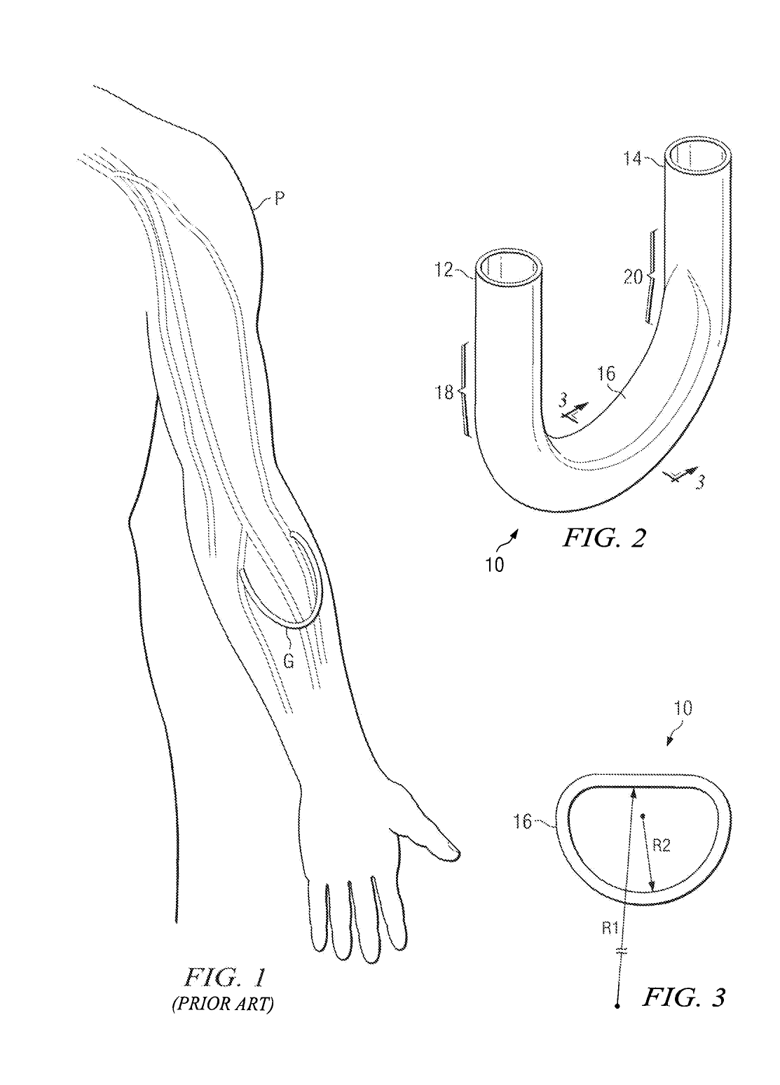

### DETAILED DESCRIPTION

The hemodialysis graft of the present invention is illustrated in FIGS. 2 and 3. A graft 10 comprising the invention has opposite ends 12 and 14 which are round in cross section. Between the ends 12 and 14 the graft 10 comprises a curved section 16 having the D-shaped cross sectional configuration illustrated in FIG. 3. The round cross sections comprising the ends 12 and 14 of the graft 10 transition to the D-shaped cross sectional illustrated in FIG. 3 in transition zones 18 and 20.

Referring specifically to FIG. 3, the radius R1 defining the inside surface of the graft 10 in the curved section 16 is relatively large as compared with the radius R2 of the outside surface of the graft 10 in the curved section 16. The D-shaped cross section of the graft 10 in the curved section 16 thereof minimizes the differential in velocities as between the inner interior surface and the outer interior surface of the graft 10 in the curved section 16 thereby minimizing sheer forces and maintaining laminar flow.

By maintaining laminar flow in blood flowing through the curved section 16 of the graft 10 cellular proliferation and coagulation of the blood is substantially reduced. Reduction in cellular proliferation and coagulation substantially extends the useful life of the graft 10. This in turn substantially reduces the number of surgeries that will be required during hemodialysis treatment of the patient.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. For use in a hemodialysis graft having at least one curved section, the: improvement comprising:
the curved section of the graft having an inner interior surface comprising a relatively large radius and an outer interior surface comprising a relatively small radius;
the differential between the radius of the inner interior surface and the radius of the outer interior surface facilitating laminar flow of blood through the curved section of the hemodialysis graft thereby minimizing cellular proliferation and blood coagulation.

2. The improvement of claim 1 wherein the curved section is generally D-shaped.

3. For use in a hemodialysis graft having at least one curved section, the improvement comprising:
the curved section of the graft having a generally D-shaped cross sectional configuration;
the generally D-shaped cross sectional configuration of the curved section facilitating laminar flow of blood through the curved section of the hemodialysis graft thereby minimizing cellular proliferation and blood coagulation.

4. A hemodialysis graft having at least one curved section, comprising:
opposite ends having a relatively small cross sectional configuration; and
a curved section situated between the opposite ends, the curved section having a relatively large cross sectional configuration.

5. The hemodialysis graft of claim 4, wherein the opposite ends are round in cross section.

6. The hemodialysis graft of claim 5, wherein the opposite ends transition to the curved section via transition zones.

7. The hemodialysis graft of claim 6, wherein the cross sectional configuration of the curved section is generally D-shaped.

8. A hemodialysis graft having at least one curved section, comprising:
opposite ends which are round in cross section;
a curved section which is generally D-Shaped in cross section; and
transition zones situated between the opposite ends and the curved section, wherein the round cross sections of the opposite ends transition to the generally D-shaped cross section of the curved section.
